(19) United States
(12) Patent Application Publication
(10) Pub. No.: US 2001/0047100 A1
(43) Pub. Date: Nov. 29, 2001

(54) Chiral imidazoyl intermediates for the synthesis of 2-(4-imidazoyl)-cyclopropyl derivatives

(76) Inventors: Hans Joergen Kjaersgaard, Farum (DK); Jim Phillips, Bay Village, OH (US); Syed M. Ali, Solon, OH (US)

Correspondence Address: ROCKEY, MILNAMOW & KATZ, LTD., TWO PRUDENTIAL PLAZA, STE. 4700, 180 NORTH STETSON AVENUE, CHICAGO, IL, 60601, US

(21) Appl. No.: 09/817,700

(22) Filed: Mar. 26, 2001

Related U.S. Application Data
(60) Provisional application No. 60/199,722, filed on Apr. 26, 2000.

(51) Int. Cl. C07F 9/6506; C07D 233/54; C07D 233/64

(52) U.S. Cl. 548/119; 548/300.1; 548/335.5; 548/341.5

(57) ABSTRACT

Novel intermediates useful in the preparation of optically active H₃ histamine receptor antagonist 2-(4-imidazoyl)-cyclopropyl derivatives are disclosed.

### FIELD OF THE INVENTION

Novel intermediates useful in the preparation of optically active H₃ histamine receptor antagonist 2-(4-imidazoyl)-cyclopropyl derivatives are disclosed.

### BACKGROUND OF THE INVENTION

Histamine plays a role in regulating attentiveness and cognition in the central nervous system (CNS), and histamine levels in the brain are controlled by the histamine H₃ receptor. Moreover, serotonin, norepinephrine, dopamine and acetylcholine all have been demonstrated to be regulated by the histamine H₃ receptor. These neurotransmitters are known to play a role in many CNS psychiatric disorders involving higher cognitive function and/or emotion. Consequently, compounds affecting H₃ receptor function could have utility in the treatment of a variety of CNS maladies, including but not limited to dementias, attention deficit hyperactivity disorder, depression, anxiety and schizophrenia.

Histamine is also involved in the control of sleep/wake states and appetite. Accordingly, histamine H₃ receptor ligands might be expected to be useful in treating insomnia, narcolepsy, age-related sleep disorders, obesity and anorexia. Although they exist in low density outside of the brain, histamine H₃ receptors are found on the sympathetic and parasympathetic nerve terminals in the periphery, including the vasculature and heart. Thus, compounds that alter histamine H₃ receptor activity might also have clinical utility in treating conditions such as migraine and cardiac dysfunction.

Imidazole compounds are disclosed in U.S. Pat. Nos. 5,559,113; 5,990,317; 6,008,240 and 5,652,258. A synthetic procedure for making 2-(4-imidazolyl)-cyclopropylamine is disclosed in Burger et al., J. Med. Chem. 1976, 19, 923. 1H-4(5)-substituted imidazole derivatives are also disclosed in WO 96/40126. Key intermediates for the preparation of potent and chiral histamine H₃ receptor agents are disclosed in Khan et al., Bioorganic & Medicinal Chemistry Letters, Vol. 7, No. 23, pp.3017-3022.

There is a need for new and improved procedures for the preparation of imidazole compounds which have utility as histamine H₃ receptor agents. It is therefore an object of the present invention to provide novel intermediates which may be used to synthesize the above-mentioned agents.

### BRIEF SUMMARY OF THE INVENTION

The invention is directed to novel compounds of Formula I as follows

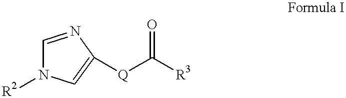

wherein Q is —C(R⁴)═C(R⁵)— or

[chemical structure]

R¹, R⁴ and R⁵ at each occurrence are independently selected from the group consisting of hydrogen, halogen, hydroxyl, alkyl, alkenyl, alkynyl, alkoxy, alkenoxy, alkynoxy, thioalkoxy, aliphatic acyl, —CF₃, nitro, amino, cyano, —N(C₁-C₃ alkyl)-C(O)(C₁-C₃ alkyl), —C₁-C₃ alkylamino, alkenylamino, alkynylamino, di(C₁-C₃ alkyl)amino, —C(O)O-(C₁-C₃ alkyl), —C(O)NH-(C₁-C₃ alkyl), —CH═NOH, —PO₃H₂, —OPO₃H₂, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, alkylaryl, aralkenyl, aralkyl, alkylheterocyclyl, heterocyclylalkyl, sulfonyl, sulfonamido, carbamate, aryloxyalkyl, carboxyl and —C(O)NH(benzyl);

n is an integer of zero to four;

R² is selected from the group consisting of alkyl, alkenyl, alkynyl, alkoxy, alkenoxy, alkynoxy, aliphatic acyl, —C₁-C₃ aminoalkyl, aminoalkenyl, aminoalkynyl, di(C₁-C₃ alkyl) aminoalkyl, —C(O)O-(C₁-C₃ alkyl), —C(O)NH-(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, aminoaryl, biaryl, heterocyclyl, alkylaryl, aralkenyl, aralkyl, alkylheterocyclyl, heterocyclylalkyl, carbamate, aryloxyalkyl, carboxyl, and —C(O)NH(benzyl); and,

R³ is selected from the group consisting of hydrogen, —OR⁶, —R¹³ and —NR⁷R⁸;

wherein R⁶ and R⁷ are chiral moieties;

R¹³ is a bicyclic chiral moiety;

R⁸ is selected from the group consisting of hydrogen, alkyl, alkenyl, alkynyl, alkoxy, alkenoxy, alkynoxy, aliphatic acyl, —C₁-C₃ alkylamino, alkenylamino, alkynylamino, di(C₁-C₃ alkyl)amino, —C(O)O-(C₁-C₃ alkyl), —C(O)NH-(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, heterocyclyl, alkylaryl, aralkenyl, aralkyl, alkylheterocyclyl, heterocyclylalkyl, carbamate, aryloxyalkyl, carboxyl, and —C(O)NH(benzyl);

wherein R¹, R², R³, R⁴, R⁵, R⁶, R⁷, R⁸ and R¹³ are unsubstituted or substituted with at least one electron donating or electron withdrawing group, and salts thereof,

with the proviso that when R² is —C(C₆H₅)₃, each R¹ or R⁴ and R⁵ are H, and R³ is —OR⁶, R⁶ is not —CH(CH₃)(C₂H₅);

and the proviso that when R² is —C(C₆H₅)₃, and Q is

[chemical structure]

wherein n is 4 and R¹ is H, R³ is not H or R¹³ when R¹³ is (1R)-(+)-2,10-camphorsultam.

More specifically, the compounds of this invention may be described by Formula II below

[chemical structure]

wherein R² is selected from the group consisting of alkyl, alkenyl, alkynyl, alkoxy, alkenoxy, alkynoxy, aliphatic acyl, —C₁-C₃ aminoalkyl, aminoalkenyl, aminoalkynyl, di(C₁-C₃ alkyl) aminoalkyl, —C(O)O-(C₁-C₃ alkyl), —C(O)NH-(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, aminoaryl, biaryl, heterocyclyl, alkylaryl, aralkenyl, aralkyl, alkylheterocyclyl, heterocyclylalkyl, carbamate, aryloxyalkyl, carboxyl, and —C(O)NH(benzyl);

R³ is selected from the group consisting of hydrogen, —OR⁶, —R¹³ and —NR⁷R⁸;

wherein R⁶ and R⁷ are chiral moieties;

R¹³ is a bicyclic chiral moiety;

R⁸ is selected from the group consisting of hydrogen, alkyl, alkenyl, alkynyl, alkoxy, alkenoxy, alkynoxy, aliphatic acyl, —C₁-C₃ alkylamino, alkenylamino, alkynylamino, di(C₁-C₃ alkyl)amino, —C(O)O-(C₁-C₃ alkyl), —C(O)NH-(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, heterocyclyl, alkylaryl, aralkenyl, aralkyl, alkylheterocyclyl, heterocyclylalkyl, carbamate, aryloxyalkyl, carboxyl and —C(O)NH(benzyl); and,

R⁹, R¹⁰, R¹¹ and R¹² are independently selected from the group consisting of hydrogen, halogen, hydroxyl, alkyl, alkenyl, alkynyl, alkoxy, alkenoxy, alkynoxy, thioalkoxy, aliphatic acyl, —CF₃, nitro, amino, cyano, —N(C₁-C₃ alkyl)—C(O)(C₁-C₃ alkyl), —C₁-C₃ alkylamino, alkenylamino, alkynylamino, di(C₁-C₃ alkyl)amino, —C(O)O-(C₁-C₃ alkyl), —C(O)NH-(C₁-C₃ alkyl), —CH═NOH, —PO₃H₂, —OPO₃H₂, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, alkylaryl, aralkenyl, aralkyl, alkylheterocyclyl, heterocyclylalkyl, sulfonyl, sulfonamido, carbamate, aryloxyalkyl, carboxyl and —C(O)NH(benzyl);

wherein R², R³, R⁶, R⁷, R⁸, R⁹, R¹⁰, R¹¹, R¹² and R¹³ are unsubstituted or substituted with at least one electron donating or electron withdrawing group,

and salts thereof,

with the proviso that when R² is —C(C₆H₅)₃, R⁹, R¹⁰, R¹¹ and R¹² are H, and R³ is —OR⁶, R⁶ is not —CH(CH₃)(C₂H₅);

and the proviso that when R² is —C(C₆H₅)₃, R⁹, R¹⁰, R¹¹ and R¹² are H, R³ is not H or R¹³ when R¹³ is (1R)-(+)-2,10-camphorsultam.

For Formula II, R² may be alkyl or aryl; and R⁹, R¹⁰, R¹¹ and R¹² may each independently be hydrogen, alkyl, aryl or halogen.

Derivatives of the compounds embodied by Formulae I and II also encompass esters, carbamates, aminals, amides and optical isomers thereof.

For Formulae I or II, chiral moieties R⁶ or R⁷ may be amines. A presently preferred compound is N-((1S)-1-Phenylethyl)[(2S,1R)-2-(1-triphenylmethylimidazol-4-yl)cyclopropyl]carboxamide.

### DETAILED DESCRIPTION OF THE INVENTION

### Definitions of Terms

The term “alkyl” as used herein alone or in combination refers to C₁-C₁₂ straight or branched, substituted or unsubstituted saturated chain radicals derived from saturated hydrocarbons by the removal of one hydrogen atom, unless the term alkyl is preceded by a Cₓ-C_{y} designation. Representative examples of alkyl groups include methyl, ethyl, n-propyl, iso-propyl, n-butyl, sec-butyl, iso-butyl, and tert-butyl among others.

The term “alkenyl”, alone or in combination, refers to a substituted or unsubstituted straight-chain or substituted or unsubstituted branched-chain alkenyl radical containing from 2 to 10 carbon atoms. Examples of such radicals include, but are not limited to, ethenyl, E- and Z-pentenyl, decenyl and the like.

The term “alkynyl”, alone or in combination, refers to a substituted or unsubstituted straight or substituted or unsubstituted branched chain alkynyl radical containing from 2 to 10 carbon atoms. Examples of such radicals include, but are not limited to ethynyl, propynyl, propargyl, butynyl, hexynyl, decynyl and the like.

The term “lower” modifying “alkyl”, “alkenyl”, “alkynyl” or “alkoxy” refers to a C₁-C₆ unit for a particular functionality. For example lower alkyl means C₁-C₆ alkyl.

The term “aliphatic acyl” alone or in combination, refers to radicals of formula alkyl-C(O)-, alkenyl-C(O)- and alkynyl-C(O)- derived from an alkane-, alkene- or alkyncarboxylic acid, wherein the terms “alkyl”, “alkenyl” and “alkynyl” are as defined above. Examples of such aliphatic acyl radicals include, but are not limited to, acetyl, propionyl, butyryl, valeryl, 4-methylvaleryl, acryloyl, crotyl, propiolyl and methylpropiolyl, among others.

The term “cycloalkyl” as used herein refers to an aliphatic ring system having 3 to 10 carbon atoms and 1 to 3 rings, including, but not limited to cyclopropyl, cyclopentyl, cyclohexyl, norbornyl, and adamantyl among others. Cycloalkyl groups can be unsubstituted or substituted with one, two or three substituents independently selected from lower alkyl, haloalkyl, alkoxy, thioalkoxy, amino, alkylamino, dialkylamino, hydroxy, halo, mercapto, nitro, carboxaldehyde, carboxy, alkoxycarbonyl and carboxamide. “Cycloalkyl” includes cis or trans forms. Furthermore, the substituents may either be in endo or exo positions in the bridged bicyclic systems.

The term “cycloalkenyl” as used herein alone or in combination refers to a cyclic carbocycle containing from 4 to 8 carbon atoms and one or more double bonds. Examples of such cycloalkenyl radicals include, but are not limited to, cyclopentenyl, cyclohexenyl, cyclopentadienyl and the like. The term “cycloalkylalkyl” as used herein refers to a cycloalkyl group appended to a lower alkyl radical, including, but not limited to cyclohexylmethyl.

The term “halo” or “halogen” as used herein refers to I, Br, Cl or F.

The term “haloalkyl” as used herein refers to a lower alkyl radical, to which is appended at least one halogen substituent, for example chloromethyl, fluoroethyl, trifluoromethyl and pentafluoroethyl among others.

The term “alkoxy”, alone or in combination, refers to an alkyl ether radical, wherein the term “alkyl” is as defined above. Examples of suitable alkyl ether radicals include, but are not limited to, methoxy, ethoxy, -propoxy, iso-propoxy, n-butoxy, iso-butoxy, sec-butoxy, tert-butoxy and the like.

The term “alkenoxy”, alone or in combination, refers to a radical of formula alkenyl-O-, provided that the radical is not an enol ether, wherein the term “alkenyl” is as defined above. Examples of suitable alkenoxy radicals include, but are not limited to, allyloxy, E- and Z- 3-methyl-2-propenoxy and the like.

The term “alkynoxy”, alone or in combination, refers to a radical of formula alkynyl-O-, provided that the radical is not an -ynol ether. Examples of suitable alkynoxy radicals include, but are not limited to, propargyloxy, 2-butynyloxy and the like.

The term “carboxyl” as used herein refers to a carboxylic acid radical, —C(O)OH.

The term “thioalkoxy”, refers to a thioether radical of formula alkyl-S-, wherein “alkyl” is as defined above.

The term “carboxaldehyde” as used herein refers to —C(O)R wherein R is hydrogen.

The term “carboxamide” as used herein refers to —C(O)NRₐR_{b} wherein Rₐ and R_{b} are each independently hydrogen, alkyl or any other suitable substituent.

The term “alkoxyalkoxy” as used herein refers to R_{c}O—R_{d}O— wherein R_{c} is lower alkyl as defined above and R_{d} is alkylene wherein alkylene is —(CH₂)_{n′}— wherein n′ is an integer from 1 to 6. Representative examples of alkoxyalkoxy groups include methoxymethoxy, ethoxymethoxy, t-butoxymethoxy among others.

The term “alkylamino” as used herein refers to RₑNH— wherein Rₑ is a lower alkyl group, for example, ethylamino, butylamino, among others.

The term “alkenylamino” alone or in combination, refers to a radical of formula alkenyl-NH- or (alkenyl)₂N-, wherein the term “alkenyl” is as defined above, provided that the radical is not an enamine. An example of such alkenylamino radical is the allylamino radical.

The term “alkynylamino”, alone or in combination, refers to a radical of formula alkynyl-NH- or (alkynyl)₂N- wherein the term “alkynyl” is as defined above, provided that the radical is not an amine. An example of such alkynylamino radicals is the propargyl amino radical.

The term “dialkylamino” as used herein refers to R_{f}R_{g}N- wherein R_{f} and R_{g} are independently selected from lower alkyl, for example diethylamino, and methyl propylamino, among others.

The term “amino” as used herein refers to H₂N—.

The term “alkoxycarbonyl” as used herein refers to an alkoxyl group as previously defined appended to the parent molecular moiety through a carbonyl group. Examples of alkoxycarbonyl include methoxycarbonyl, ethoxycarbonyl, and isopropoxycarbonyl among others.

The term “aryl” or “aromatic” as used herein alone or in combination refers to a substituted or unsubstituted carbocyclic aromatic group having about 6 to 12 carbon atoms such as phenyl, naphthyl, indenyl, indanyl, azulenyl, fluorenyl and anthracenyl; or a heterocyclic aromatic group selected from the group consisting of furyl, thienyl, pyridyl, pyrrolyl, oxazolyl, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthridinyl, pteridinyl, carbazolyl, acridinyl, phenazinyl, phenothiazinyl, phenoxyazinyl, pyrazolo[1,5-c]triazinyl and the like. “Arylalkyl” and “alkylaryl” employ the term “alkyl” as defined above. Rings may be multiply substituted.

The term “aralkyl”, alone or in combination, refers to an aryl substituted alkyl radical, wherein the terms “alkyl” and “aryl” are as defined above. Examples of suitable aralkyl radicals include, but are not limited to, phenylmethyl, phenethyl, phenylhexyl, diphenylmethyl, pyridylmethyl, tetrazolyl methyl, furylmethyl, imidazolyl methyl, indolylmethyl, thienylpropyl and the like.

The term “aralkenyl”, alone or in combination, refers to an aryl substituted alkenyl radical, wherein the terms “aryl” and “alkenyl” are as defined above.

The term “arylamino”, alone or in combination, refers to a radical of formula aryl-NH-, wherein “aryl” is as defined above. Examples of aminoaryl radicals include, but are not limited to, phenylamino(anilido), naphthlamino, 2-, 3-, and 4-pyridylamino and the like.

The term “biaryl”, alone or in combination, refers to a radical of formula aryl-aryl, wherein the term “aryl” is as defined above.

The term “thioaryl”, alone or in combination, refers to a radical of formula aryl-S-, wherein the term “aryl” is as defined above. An example of a thioaryl radical is the thiophenyl radical.

The term “aroyl”, alone or in combination, refers to a radical of formula aryl-CO-, wherein the term “aryl” is as defined above. Examples of suitable aromatic acyl radicals include, but are not limited to, benzoyl, 4-halobenzoyl, 4-carboxybenzoyl, naphthoyl, pyridylcarbonyl and the like.

The term “heterocyclyl”, alone or in combination, refers to a non-aromatic 3- to 10-membered ring containing at least one endocyclic N, O, or S atom. The heterocycle may be optionally aryl-fused. The heterocycle may also optionally be substituted with at least one substituent which is independently selected from the group consisting of hydrogen, halogen, hydroxyl, amino, nitro, trifluoromethyl, trifluoromethoxy, alkyl, aralkyl, alkenyl, alkynyl, aryl, cyano, carboxy, carboalkoxy, carboxyalkyl, oxo, arylsulfonyl and aralkylaminocarbonyl among others.

The term “alkylheterocyclyl” as used herein refers to an alkyl group as previously defined appended to the parent molecular moiety through a heterocyclyl group.

The term “heterocyclylalkyl” as used herein refers to a heterocyclyl group as previously defined appended to the parent molecular moiety through an alkyl group.

The term “aminal” as used herein refers to a hemi-acetal of the structure RₕC(NRᵢRⱼ)(NRₖRₗ)- wherein Rₕ, Rᵢ, Rⱼ, Rₖ and Rₗ are each independently hydrogen, alkyl or any other suitable substituent

Use of the above terms is meant to encompass substituted and unsubstituted moieties. Substitution may be by one or more groups such as alcohols, ethers, esters, amides, sulfones, sulfides, hydroxyl, nitro, cyano, carboxy, amines, heteroatoms, lower alkyl, lower alkoxy, lower alkoxycarbonyl, alkoxyalkoxy, acyloxy, halogens, trifluoromethoxy, trifluoromethyl, alkyl, aralkyl, alkenyl, alkynyl, aryl, cyano, carboxy, carboalkoxy, carboxyalkyl, cycloalkyl, cycloalkylalkyl, heterocyclyl, alkylheterocyclyl, heterocyclylalkyl, oxo, arylsulfonyl and aralkylaminocarbonyl or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)O—. Rings may be substituted multiple times.

The terms “electron-withdrawing” or “electron-donating” refer to the ability of a substituent to withdraw or donate electrons relative to that of hydrogen if hydrogen occupied the same position in the molecule. These terms are well-understood by one skilled in the art and are discussed in Advanced Organic Chemistry by J. March, 1985, pp. 16-18, incorporated herein by reference. Electron withdrawing groups include halo, nitro, carboxyl, lower alkenyl, lower alkynyl, carboxaldehyde, carboxyamido, aryl, quaternary ammonium, trifluoromethyl, and aryl lower alkanoyl among others. Electron donating groups include such groups as hydroxy, lower alkyl, amino, lower alkylamino, di(lower alkyl)amino, aryloxy, mercapto, lower alkylthio, lower alkylmercapto, and disulfide among others. One skilled in the art will appreciate that the aforesaid substituents may have electron donating or electron withdrawing properties under different chemical conditions. Moreover, the present invention contemplates any combination of substituents selected from the above-identified groups.

The most preferred electron donating or electron withdrawing substituents are halo, nitro, alkanoyl, carboxaldehyde, arylalkanoyl, aryloxy, carboxyl, carboxamide, cyano, sulfonyl, sulfoxide, heterocyclyl, guanidine, quaternary ammonium, lower alkenyl, lower alkynyl, sulfonium salts, hydroxy, lower alkoxy, lower alkyl, amino, lower alkylamino, di(lower alkyl)amino, amine lower alkyl mercapto, mercaptoalkyl, alkylthio and alkyldithio.

As used herein, the term “composition” is intended to encompass a product comprising the specified ingredients in the specified amounts, as well as any product which results, directly or indirectly, from a combination of the specified ingredients in the specified amounts.

The term “chiral moieties” as used herein refers to substituents having a chiral center. Preferred chiral moieties are amines or bicyclic compounds. Examples of suitable chiral amines include chiral alkyl amines such as (+)- or (−)-2-aminobutane and (+)- or (−)-2-aminoheptane; chiral hydroxy-substituted alkylamines such as (+)- or (−)-2-amino-1-butanol, (+)- or (−)-2-amino-1-propanol, (+) or (−)-2-amino-3-methyl-1-butanol, (+)- or (−)-leucinol, and (+)-isoleucinol; chiral phenyl or naphthyl-substituted alkyl amines such as (+)- or (−)-alpha methyl benzylamine; (+)- or (−)-alpha-(1-naphthyl)ethylamine, and (+)-or (−)-alpha-(2-naphthyl)ethylamine; chiral alkylamines substituted with both hydroxy and phenyl such as (+)- or (−)-2-phenylglycinol, (+)- or (−)-threo-2-amino-1-phenyl-1,3-propanediol, (+)- or (−)-norephedrin, (+)- or (−)-2-amino-3-phenyl-1-propanol and (+)- or (−)-2-amino-1,2-diphenylethanol; and other substituted alkylamines such as (+)- or (−)-alpha-methyl-p-nitrobenzylamine, (+)- or (−)-threo-2-amino-1-(4-nitrophenyl)-1,3-propanediol, (+)- or (−)-norepinephrine, (+)-dehydroabietylamine, (+)-2-amino-3-methoxy-1-phenylpropanol, L-tyrosinol and lower alkyl esters of alpha amino acids such as (+)- or (−)-alanine, (+)- or (−)-valine, (+)- or (−)-leucine, (+)- or (−)-isoleucine, (+)- or (−)-phenylalanine, (+)- or (−)-tyrosine, (+)- or (−)-serine, and (+)- or (−)-threonine. Examples of suitable chiral compounds include benzylamine and derivatives thereof. An example of a suitable bicyclic chiral compound is (1R)-(+)-2,10-camphorsultam. When amine chiral compounds are utilized to make the present imidazoyl derivatives, the chiral moiety is attached through the nitrogen group. A preferred benzylamine is of the structure shown below.

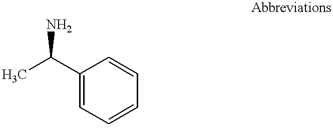

Abbreviations which have been used in the schemes and the examples which follow are: EtOAc for ethyl acetate; DMF for dimethylformamide; THF for tetrahydrofuran; TEA for triethylamine; DMSO for dimethylsulfoxide; DME for dimethoxyethane, and DIBAL-H for diisobutyl aluminum hydride.

Scheme I shown below, illustrates the synthesis of compound 1, which is utilized in the synthesis of the present compounds.

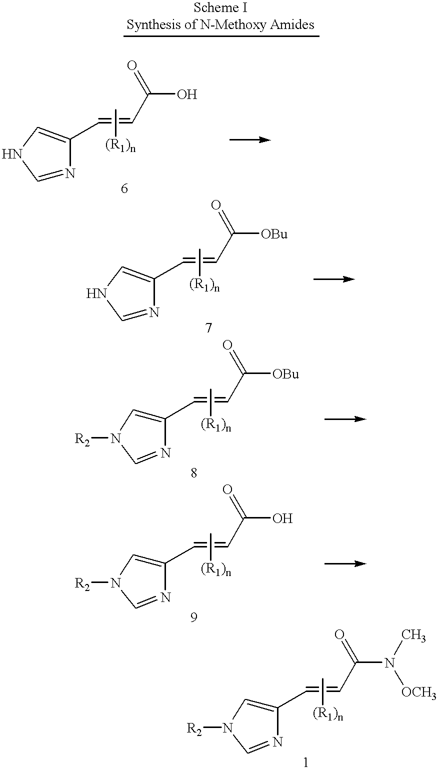

The cyclopropyl imidazoles of this invention may be synthesized according to the general synthesis depicted in Scheme II.

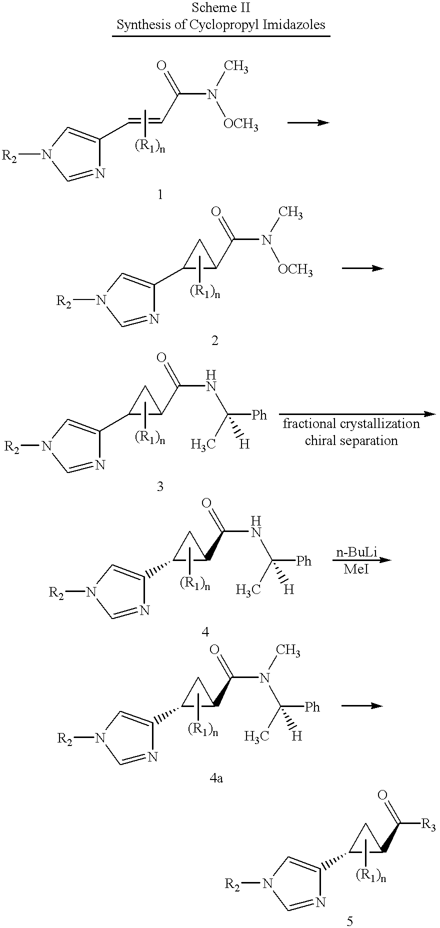

An appropriately substituted urocanic acid 6 is converted to an N-methoxy amide 1 in four steps (Scheme I), followed by reacting the resulting olefinic amide 1 with trimethylsulfoxonium iodide in the presence of a base such as tert-butoxide to give rise to the corresponding cyclopropyl amide 2 (Scheme II). Hydrolysis of 2 under basic conditions affords the corresponding acid, which is in turn converted to the chiral amide 3. Fractional crystallization of the racemic mixture yields the pure enantiomer 4 which may be transformed into an aldehyde or an acid 5 (R₃=H or OH respectively).

Antagonists can then be synthesized from 5, as shown in Scheme III below.

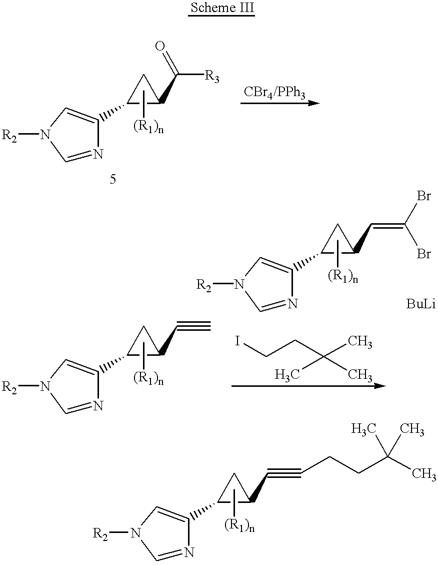

A detailed description of the preparation of representative compounds of the present invention is set forth in the Examples.

The compounds of the present invention can be used in the form of salts derived from inorganic or organic acids. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzene sulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluene sulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a metal cation or with ammonia or an organic primary, secondary or tertiary amine. Salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethyl ammonium, tetraethyl ammonium, methyl ammonium, dimethyl ammonium, trimethyl ammonium, triethyl ammonium, diethyl ammonium, and ethyl ammonium among others. Other representative organic amines useful for the formation of base addition salts include ethylenediamine, ethanolamine, diethanolamine, piperidine, piperazine and the like.

The present invention contemplates both synthetic compounds of Formulae I and II of the present invention, as well as compounds formed by in vivo conversion to compounds of the present invention.

Compounds of the present invention may exist as stereoisomers wherein asymmetric or chiral centers are present. These stereoisomers are “R” or “S” depending on the configuration of substituents around the chiral carbon atom. The present invention contemplates various stereoisomers and mixtures thereof. Stereoisomers include enantiomers and diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The Examples which follow are presented to describe preferred embodiments and utilities of the invention and are not meant to limit the invention unless otherwise stated in the claims appended hereto.

### EXAMPLE 1

N-Methoxy-N-methyl[2-(1-triphenylmethylimidazol-4-yl)cyclopropyl]carboxamide 10, was synthesized in the following manner.

First, n-butyl (2E)-3-(imidazol-4-yl)prop-2-enoate 11 was prepared in the following manner. A mixture of n-butanol (11 L) and urocanic acid (2.988 kg, 21.6 mol) was heated at 60-80° C. while p-toluene sulfonic acid monohydrate (4.5 kg, 23.7 mol) was added in 5 portions over a period of 1 hour. Next, the mixture was heated to reflux and water was removed from the reaction mixture by azeotropic distillation with n-butanol at atmospheric pressure. After 2 hours (approximately 4 L distillate collected), additional n-butanol (2 L) was added to the mixture and distillation was continued for another 1.5 hours (approximately 8.5 L distillate collected). Distillation was continued at reduced pressure until approximately 13 L of distillate was collected. Then toluene (16 L) was slowly added to the residue while keeping the temperature at 80-100° C. The mixture was then allowed to cool to room temperature over a period of 12 hours, giving rise to a suspension which was filtered. Then the solid obtained was washed with toluene (6 L) and dried in a tray dryer at 50° C. to give the p-toluene sulfonic acid salt of n-butyl urocanate as an off-white solid (7.4 kg, 93%), which was mixed with water (45 L). The pH of the resulting mixture was adjusted to 10.5 with aqueous sodium hydroxide (27.65%, approximately 3.0 kg) at 10-15° C. The mixture was then extracted twice with CH₂Cl₂ (27 L+8 L). The combined organic extracts were washed with water (10 L), dried over Na₂SO₄ and filtered. The filtrate was concentrated to approximately 15 L at atmospheric pressure to remove traces of water to give a residue of compound 11.

Next, n-butyl (2E)-3-(1-triphenylmethylimidazol-4-yl)prop-2-enoate 12 was prepared as follows. To the residue 11, CH₂Cl₂ (25 L) was added, followed by the addition of Et₃N (2.2 kg, 21.8 mol). Triphenylmethyl chloride (5.7 kg, 20.4 mol) was added to the mixture, which had been cooled at 8-12° C., in six portions over a period of approximately 30 minutes. The resulting mixture was stirred at 10-15° C. for 30 minutes. Water (15 L) was then added to the mixture and the organic layer was separated, dried over Na₂SO₄, and filtered. Next, the filtrate was concentrated to a volume of approximately 29 L. Heptanes (bp 80-100° C., 50 L) were then added over a period of approximately 30 minutes, and the resulting suspension was cooled to −5 to 0° C. The solid was isolated on a centrifuge and washed with heptanes (10 L), then dried in a tray dryer at 50° C. to give compound 12 (8.1 kg, 85% from urocanic acid).

(2E)-3-(1-Triphenylmethylimidazol-4-yl)prop-2-enoic acid, 13, was then prepared as follows. A mixture of 12 (11.2 kg, 25.7 mol) was heated to 45-50° C. Then a solution of KOH (5.6 kg) in water (42 L) was added and the resulting mixture was heated to 60-65° C. and stirred at this temperature for 1.5 hours. After cooling to approximately 40° C., and acidifying to pH 3.3 by adding aqueous HCl (12.3 %, approximately 26 kg), the resulting suspension was diluted by water (35 L), cooled to 20-25° C., and filtered. The solid was then washed with water (3×10 L) and dried in a tray dryer at 70° C. to afford 13 (9.7 kg, 99%) as an off-white solid.

(2E)-N-Methoxy-N-methyl-3-(1-triphenylmethylimidazol-4-yl)prop-2-enamide 14 was then prepared as follows. To a mixture of 13 (8.0 kg, 21.0 mol), triethylamine (2.35 kg, 23.3 mol) and CH₂Cl₂ (105 kg), under a nitrogen atmosphere, cooled at −5-0° C., was added isobutyl chloroformate (3.15 kg, 23.1 mol). After stirring was continued at −5-0° C. for 30 minutes, N,O-dimethylhydroxylamine hydrochloride (2.25 kg, 23.1 mol) was added followed by triethylamine (2.35 kg, 23.3 mol) at −5-0° C. The mixture was then stirred at 10° C. for 3 hours and cooled to −5-0° C. Additional amounts of triethylamine (260 g, 2.6 mol) and isobutyl chloroformate (350 g, 2.6 mol) were added at −5-10° C., and after 15 minutes of stirring, more triethylamine (260 g, 2.6 mol) and N, O-dimethylhydroxylamine hydrochloride (250 g, 2.6 mol) were added at −5-0° C. The mixture was allowed to warm to room temperature and stirred overnight. After adding water (15 L), the organic phase was separated, dried over Na₂SO₄ with the addition of triethylamine (0.4 kg), and filtered through a silica gel pad (10 kg). Next, the filtered cake was washed successively with CH₂Cl₂ (18 L), a solution of EtOAc: CH₂Cl₂ (4:1; 20 L), EtOAc: CH₂Cl₂ (9:1; 20 L), and EtOAc (20 L). The combined filtrates were concentrated at atmospheric pressure to approximately 25 L. EtOAc (38 L) and heptanes (48 L) were added at 65-75° C., and the resulting suspension was allowed to cool to room temperature overnight. A solid was isolated on a centrifuge and washed with heptanes:EtOAc (4:1; 2×9 L) and dried in a tray dryer at 70° C. to give 14 (7.9 kg, 89%).

Lastly, N-Methoxy-N-methyl [2-(1-triphenylmethylimidazol-4-yl)cyclopropyl]carboxamide 10 was prepared as follows. A mixture of trimethylsulfoxonium iodide (4.95 kg, 22.5 mol), potassium tert-butoxide (2.55 kg, 22.7 mol), and DMSO (56 kg) under a nitrogen atmosphere was stirred for 30 minutes at room temperature. Compound 14 (7.9 kg, 18.7 mol) was added and the resulting mixture was stirred at 0-55° C. for 5 hours, allowed to cool to room temperature overnight, and diluted with water (115 kg) at 25-30° C. The solid product was isolated on a centrifuge, washed with water (55 kg), and dried in a tray dryer at 70° C. to give 10 as an off-white solid (7.75 kg, 95%).

### EXAMPLE 2

2-(1-Triphenylmethylimidazol-4-yl)cyclopropanecarboxylic acid 15, was synthesized in the following manner. To a mixture of tetrahydrofuran (51.5 kg) and water (652 g), potassium tert-butoxide (13.3 kg mol) was added in three portions while keeping the temperature at 20-30° C. Compound 10 (7.7 kg, 17.6 mol) was then added in four portions while keeping the temperature at 20-25° C. After stirring the mixture at 20-25° C. for 1.5 hours, it was diluted with water (26 kg) over a period of 15 minutes, and concentrated under reduced pressure until 60 L of distillate was collected. After diluting with ethanol (30 kg), the pH of the solution was adjusted to 4.4 with aqueous HCl (3 N, approximately 39.5 L) at 30-35° C. The resulting suspension was cooled to 20° C., stirred at that temperature for 30 minutes, and centrifuged to provide a solid which was washed with water (50 L) and dried in a tray dryer at 70° C. to give 15 (6.4 kg, 92%).

### EXAMPLE 3

N-((1S)-1-Phenylethyl)[(2S,1R)-2-(1-triphenylmethylimidazol-4-yl)cyclopropyl]carboxamide 16, was prepared in the following manner. To a mixture of compound 15 (6.4 kg, 16.2 mol) and triethylamine (1.80 kg, 17.8 mol) in methylene chloride (78 kg) at 0-5° C., was added isobutyl chloroformate (2.43 kg, 17.8 mol) with stirring. After 30 minutes, (R)-(+)-α-methylbenzylamine (available from Aldrich) was added at 0-5° C. over a period of approximately 30 minutes. Stirring was then continued at this temperature for another hour, after which water (20 L) was added to the mixture and the organic phase was separated, dried over Na₂SO₄, and filtered. The filtrate was concentrated first at atmospheric pressure, and later at reduced pressure to remove methylene chloride from the mixture. Methanol (41 kg) was added to the residue and the resulting mixture was cooled to approximately 20° C. and seeded. After crystallization started, the mixture was slowly heated to 50° C. and then allowed to cool to 26-28° C. overnight. Next, the suspension was filtered and the crystals were washed with MeOH (15 L, 25° C.). The solid was then dried in a tray dryer at 70° C. to give compound 16 as an off-white solid (2.3 kg, 58%).

### EXAMPLE 4

(2S,1R)-2-(1-Triphenylmethylimidazol-4-yl)cyclopropanecarbaldehyde 17, was prepared as follows. A mixture of compound 16 (1090 g, 2.19 mol) and toluene (7.5 L) was heated to reflux and concentrated to remove traces of protic solvents. After collecting approximately 5.5 L of distillate, the distillation was stopped and the residue was cooled to approximately 60° C. under a nitrogen atmosphere. Tetrahydrofuran (7.5 L) was then added to give a clear solution. Next, the solution was transferred into a 20 L stainless steel reactor and cooled to approximately −50° C. by means of dry ice/acetone under a nitrogen atmosphere. After adding butyl lithium in hexanes (15%, 960 g, 2.25 mol) over a period of 30 minutes at −50° C., the mixture was stirred for 10 minutes, followed by the addition of methyl iodide (342 g, 2.41 mol) in one portion. The resulting mixture was stirred overnight under nitrogen at room temperature.

Into a separate flask, containing a mixture of tetrahydrofuran (2 L) and DIBAL in toluene (25%, 1850 g, 3.25 mol) at 0° C. under a nitrogen atmosphere, was added butyllithium in hexanes (15%, 1390 g, 3.25 mol) over a period of 1 hour. The resulting mixture was stirred at this temperature for another 30 minutes, and was then added to the above reaction mixture at 15-20° C. over a period of 2 hours. After stirring at room temperature overnight, the mixture was cooled to 5-10° C. Then an aqueous solution of NaCl (20%, 2 L) was slowly added to give a mixture of two phases, which were separated. Silica gel (800 g) and celite (500 g) were then added to the organic phase with stirring. The suspension was filtered through a silica gel pad (1600 g) and the filter cake was washed with a mixture of toluene:EtOAc (3:1; 13 L). The filtrate was concentrated using a rotary evaporator to give the crude compound 17 (995 g) as a sticky off-white solid.

### EXAMPLE 5

2-(1-Triphenylmethyl-4-imidazolyl)-2-methyl-2-cyclopropylcarboxylic acid, 18, was synthesized in the following manner.

First, butyl 2-(1-triphenylmethyl-4-imidazol)-2-methyl-2-cyclopropylcarboxylate, 19, was synthesized in the following manner.

To a mixture of Me₂SO⁺ I⁻ (2.4 mmol) and NaH (60% in mineral oil, 2.4 mmol) was added dropwise with stirring under nitrogen dry DMSO (10 ml), and the resulting mixture was stirred for 30 minutes. After the addition of 3-(1-triphenylmethyl-5-imidazolyl)-2-methyl-2-propenyl-butyl ester (1.0 g, 2.22 mmol) in dry DMSO/THF (20 ml, 1/1), the reaction mixture was heated at 60° C. for 24 hours, cooled, poured into cold 1 M HCl (25 ml), and extracted with ether (2×100 ml). The ether layer was then dried (MgSO₄) filtered, and evaporated in vacuo. The residue was purified by flash chromatography using ethyl acetate/hexanes:4/6 to afford compound 19 (0.85 g).

Next, 2-(1-triphenylmethyl-4-imidazolyl)-2-methyl-2-cyclopropylcarboxylic acid 18 was prepared as follows. To a solution of compound 19 (1.4 g, 30 mmol) in EtOH (15 ml), aqueous KOH (12%, 15 ml) was added in portions with stirring at 40-50° C. After stirring at 60° C. for 24 hours, the solution was cooled, diluted with water (30 ml), extracted with ether and then acidified to pH=6.0 with 0.5 M HCl. The precipitate obtained was filtered off and dried to give compound 18.

### EXAMPLE 6

tert-Butyl 2-(1-triphenylmethylimidazol-4-yl)-3-methylcyclopropanecarboxylate 20, was synthesized in the following manner. A solution of diphenylethylsulphonium tetrafluoroborate (10.39 g, 34.38 mmol) and dry CH₂Cl₂ (2.72 ml, 42.50 mmol) in anhydrous DME (300 ml) was cooled to −70° C. under N₂. In a separate flask, lithium diisopropylamide (LDA) was prepared by the addition of n-BuLi (13.6 ml, 2.5 M in hexanes, 34.0 mmol) to neat diisopropylamine (4.78 ml, 34.0 mmol) at 0° C. under N₂. After 30 minutes, dry DME (50 ml) was added to the solid LDA and the resulting solution was cooled to −78° C. The DME solution of LDA was added via cannula to the DME solution of diphenylethylsulphonium tetrafluoroborate and CH₂Cl₂ to form a solution containing the corresponding ylide. After 30 minutes, a solution of tert-butyl (2E)-3-(1-triphenylmethylimidazol-4-yl) prop-2-enoate (5.0g, 11.4 mmol) in THF (50 ml) and DME (20 ml) was added via cannula to the ylide solution. The resulting mixture was stirred at −70° C. for 3 hours, allowed to slowly warm to 0° C. over 6 hours, quenched by the addition of saturated ammonium chloride (200 ml), and extracted with ethyl acetate (200 ml). The ethyl acetate layer was separated, dried (MgSO₄), filtered, and evaporated in vacuo to give a white solid which was purified by flash chromatography using ethyl acetate/hexanes: 1/9 to afford compound 20 (530 mg). NMR (¹H, CDCl₃): δ: 7.31(m, 9H), 7.11 (m, 7H), 6.55 (s, 1H), 2.50 (dd, 1H, J=4.8Hz, J=4.8 Hz), 1.68 (m, 1H), 1.59 (m, 1H), 1.41 (s, 9H), 0.96 (d, 3H, J=6.3 Hz).

### EXAMPLE 7

2-(1-Triphenylmethylimidazol-4-yl)-3-methylcyclopropanecarbaldehyde 21, was synthesized according to the following procedure.

First, [2-(1-Triphenylmethylimidazol-4-yl)-3-methylcyclopropyl]methan-1-ol 22 was prepared as follows. A solution of tert-butyl 2-(1-triphenylmethylimidazol-4-yl)-3-methylcyclopropanecarboxylate (0.42 g, 0.91 mmol) in dry THF (15 ml) was cooled to 0° C. under N₂. LiAlH₄ (0.12 g, 3.1 mmol) was added as a solid and the resulting mixture was stirred for 5 hours at room temperature. To the reaction mixture was added slowly and sequentially water (0.3 ml), 10% sodium hydroxide solution (0.3 ml), and more water (0.9 ml). The reaction mixture was filtered through a pad of celite, which was subsequently washed with ethyl acetate (50 ml). The filtrate was partitioned between water and ethyl acetate (50 ml: 50 ml). The ethyl acetate layer was separated, dried (MgSO₄), filtered, and evaporated in vacuo to give a white solid which was purified by flash chromatography, using ethyl acetate/hexanes: 6/4 to give 22 (368 mg) as a white solid. NMR (¹H, CDCl₃): δ: 7.30 (m, 9H), 7.11 (m, 7H), 6.47 (s, 1H), 3.56 (m, 2H), 1.82 (dd, 1H, J=4.8Hz, J=5.1Hz), 0.98 (m, 1H), 0.93 (m, 1H), 0.88 (d, 3H, J=6.0 Hz).

Then 2-(1-Triphenylmethylimidazol-4-yl)-3-methyl cyclopropanecarbaldehyde 21 was prepared as follows. To a solution of oxalyl chloride (0.69 ml, 1.37 mmol, 2.0M in CH₂Cl₂) in dry CH₂Cl₂ (15 ml) under N₂ at −78° C. was added dry DMSO (0.2 ml, 2.74 mmol). After 30 minutes, compound 22 (0.36 g, 0.91 mmol) in CH₂Cl₂ (5 ml) was added. Next, TEA (0.51 ml, 3.65 ml) was added after the resulting mixture was stirred for an additional 30 minutes. The reaction mixture was then warmed to −20° C., and after the addition of a saturated aqueous ammonium chloride solution (50 ml), the CH₂Cl₂ layer was separated, dried (MgSO₄), filtered, and evaporated in vacuo to give a light yellow sticky solid. Purification of the solid on a flash chromatography column using ethyl acetate/hexanes: 15/85, then 30/70 gave 21 as white foam (275 mg. 76%). NMR (¹H, CDCl₃): δ: 9.27 (d, 1H, J=4.8 Hz), 7.32 (m, 9H), 7.10 (m, 7H), 6.56 (s, 1H), 2.70 (dd, 1H, J=4.8 Hz, J =4.6 Hz), 2.05 (m, 1H), 1.82 (m, 1H), 1.06 (d, 1H, 6.3 Hz).

### EXAMPLE 8

2-(1-Triphenylmethylimidazol-4-yl)-3-methylcyclopropanecarboxylic acid 23, was synthesized according to the following procedure. To a solution of compound 21 (0.275 g, 0.70 mmol) in acetone (25 ml), was added at room temperature sequentially t-butanol (35 ml), Na₂HPO₄ (0.49M, 7 ml), and an aqueous potassium permanganate solution (5 ml, 0.6 g, 3.8 mmol). The reaction mixture was stirred at room temperature for 1.5 hours before the addition of a saturated aqueous sodium bisulfite solution (30 ml). The pH was then adjusted to 3.0 with 10% aqueous HCl, and the mixture was extracted with ethyl acetate (100 ml). The organic layer was separated, dried (MgSO₄), filtered, and evaporated in vacuo to give 23 (335 mg) as a white foam. NMR (¹H, CDCl₃): δ: 7.88 (s, 1H), 7.36 (m, 1H), 7.06 (m, 6H), 6.61 (s, 1H), 2.57 (dd, 1H, J=4.8 Hz, J=3.9 Hz), 1.91 (m, 1H), 1.81 (m, 1H), 0.94 (d, 3H, J=6.3 Hz).

### EXAMPLE 9

2-(1-Triphenylmethylimidazol-4-yl)-1-methylcyclopropanecarbaldehyde 24, was synthesized in the following manner.

First, (2E)-3-(1-Triphenylmethylimidazol-4-yl)-2-methylpropanol 25 was prepared as follows. To a solution of ethyl (2E)-3-(1-triphenylmethylimidazol-4-yl)-2-methylprop-2-enoate (6.0 g, 14.2 mmol) in dry THF (120 ml) under N₂ at −78° C., was added DIBAL-H (1.0M in toluene, 70 ml, 70 mmol) dropwise via syringe. After the addition was complete, the reaction mixture was stirred for an additional hour, then quenched by the dropwise addition of methanol (8 ml), followed by the addition of an aqueous solution of potassium tartrate along with ethyl acetate (200 ml). The mixture was stirred overnight and then the organic layer separated, dried (MgSO₄), filtered, and evaporated in vacuo to give 25 (5.2 g) as a fluffy white solid, used without further purification. NMR (¹H, CDCl₃): δ: 7.43 (s,1H), 7.31 (m,9H), 7.13 (m,6H), 6.74 (s,1H), 6.37 (s,1H), 4.11 (s,2H), 1.90 (s,3H).

Next, (2E)-3-(1-Triphenylmethylimidazol-4-yl)-2-methyl-1-(tert-butyldiphenylsiloxy)-propane 26 was prepared as follows. Compound 25 (2.0 g, 5.26 mmol) was dissolved in dry DMF (30 ml) and the solution cooled to 0° C. under N₂. Imidazole (0.43 g, 6.3 mmol) was added followed by tert-butyldiphenylsilyl chloride (1.64 ml, 6.32 mmol). The reaction was warmed to room temperature and stirred overnight. Next, saturated sodium chloride solution was added (100 ml) and the resulting mixture was extracted with ethyl acetate (2×100 ml). The ethyl acetate layer was separated, washed with saturated sodium chloride solution (3×50 ml), dried (MgSO₄), filtered, and evaporated in vacuo. Purification by flash chromatography using ethyl acetate/hexanes: 5/95 gave 26 (2.6 g) as a white solid. NMR (¹H, CDCl₃): δ: 7.65 (m, 4H), 7.35(m, 14H), 7.13 (m, 8H), 6.74 (s, 1H), 6.49 (s, 1H), 4.14 (s, 2H), 1.80 (s, 3H), 1.05 (s, 9H).

Next, 1-[2-(1-Triphenylmethylimidazol-4-yl)- 1-methylcyclopropyl]-1-(tert-butyldiphenylsiloxy)-methane 27 was synthesized according to the following procedure. Compound 26 (0.64 g, 1.03 mmol) was dissolved in dry 1,2-dichloroethane (30 ml) and the resulting solution was cooled to 0° C. under N₂. To a solution of iodochloromethane (0.30 ml, 4.14 mmol) in dry 1,2-dichloroethane (20ml) at 0° C. under N₂, diethylzinc (1.0M in ether, 2.27 ml, 2.27 mmol) was added followed by the silyl ether solution via cannulation over 15 minutes. The reaction mixture was stirred for 1.5 hours, quenched by the addition of saturated ammmonium chloride (50 ml), and then extracted with ethyl acetate (100 ml). The ethyl acetate layer was separated, dried (MgSO₄), filtered, and evaporated in vacuo to give 27 (0.74 g) as a white foam which was used without further purification. NMR (¹H, CDCl₃): δ: 7.62 (m, 6H), 7.31(m, 12H), 7.11 (m, 6H), 7.00 (m, 2H), 6.44 (s, 1H), 3.61 (d, 1H, J=10.2 Hz), 3.48 (d, 1H, J=10.2Hz), 1.82 (m, 1H), 1.00 (s, 9H), 0.92 (s, 3H), 0.88(m, 2H). MS: (M+1)=633.23 (Electrospray)

Next, [2-(1-Triphenylmethylimidazol-4-yl)-1-methylcyclopropyl]methan-1-ol 28 was prepared in the following manner. To a solution of compound 27 (2.24 g, 3.54 mmol) in dry THF (30 ml) at room temperature under N₂, was added N-butyl ammonium fluoride (1.0M in THF, 15 ml, 15 mmol). The reaction mixture was heated at 70° C. for 3 hours, cooled, diluted with water (50 ml), and extracted with ethyl acetate (50 ml). The ethyl acetate layer was separated, dried (MgSO₄), filtered, and evaporated in vacuo to give a brown oil. Purification using flash chromatography with ethyl acetate/hexanes: 6/4 gave 28 (1.24 g) as a light yellow foam. NMR (¹H, CDCl₃): δ: 7.62 (m, 6H), 7.31(m, 12H), 7.11 (m, 6H), 7.00 (m, 2H), 6.44 (s, 1H), 3.61 (d, 1H, J=10.2 Hz), 3.48 (d, 1H, J=10.2Hz), 1.82 (m, 1H), 1.00 (s, 9H), 0.92 (s, 3H), 0.88(m, 2H).

Finally, 2-(1-Triphenylmethylimidazol-4-yl)-1-methylcyclopropanecarbaldehyde 24 was prepared as follows. To a mixture of oxalyl chloride (2.0 M in CH₂Cl₂, 1.98 ml, 3.95 mmol) in dry CH₂Cl₂ (30 ml) at −78° C. under N₂ and dry DMSO (0.56 ml, 7.89 mmol) stirred for 20 minutes, was added 28 (1.04 g, 2.63 mmol) in dry CH₂Cl₂ (15 ml). After an additional 30 minutes stirring, TEA (1.48 ml, 10.52 mmol) was added and the reaction mixture was warmed to −20° C. and then quenched by the addition of a saturated aqueous ammonium chloride solution (50 ml). The CH₂Cl₂ layer was separated, dried (MgSO₄), filtered, and evaporated in vacuo to give a white foam. Purification by flash chromatography using ethyl acetate/hexanes 2/8 gave 24 (620 mg) as a white foam. NMR (¹H, CDCl₃): δ: 8.84 (s, 1H), 7.31(m, 9H), 7.11 (m, 7H), 6.61 (s, 1H), 2.54 (m, 1H), 1.58 (m, 1H), 1.07 (s, 3H), 0.92 (m, 1H).

### EXAMPLE 10

2-(1-Triphenylmethylimidazol-4-yl)-1-methylcyclopropanecarboxylic acid 29, was synthesized according to the following procedure. To a solution of compound 24 (0.45 g, 1.15 mmol) in tert-butanol (20 ml) and acetone (10 ml) at room temperature, was added sodium dibasic buffer (0.42M, 14 ml) followed by potassium permanganate (0.6 g dissolved in 25 ml). The reaction mixture was stirred for 1 hour, and quenched by the addition of a solution of sodium hydrogen sulfite (10 ml). The pH of the mixture was adjusted to 2 with aqueous HCl (1M), and then the mixture was extracted with ethyl acetate (50 ml). The ethyl acetate layer was separated, dried (MgSO₄), filtered, and evaporated in vacuo to give compound 29 (392 mg) as a white solid. NMR (¹H, CDCl₃): δ: 7.42 (s, 1H), 7.31(m, 9H), 7.09 (m, 6H), 6.43 (s, 1H), 2.71 (m, 1H), 1.60 (m, 1H), 1.08 (s, 3H), 0.75 (m, 1H).

All references cited are hereby incorporated by reference.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made in the composition, operation and arrangement of the method of the present invention described herein without departing from the concept and scope of the invention as defined in the following claims:

We claim:

1. A compound of the structure 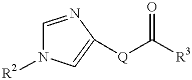
wherein Q is —C(R⁴)═C(R⁵)— or [chemical structure]
R¹, R⁴ and R⁵ at each occurrence are independently selected from the group consisting of hydrogen, halogen, hydroxyl, alkyl, alkenyl, alkynyl, alkoxy, alkenoxy, alkynoxy, thioalkoxy, aliphatic acyl, —CF₃, nitro, amino, cyano, —N(C₁-C₃ alkyl)-C(O)(C₁-C₃ alkyl), -C₁-C₃ alkylamino, alkenylamino, alkynylamino, di(CI-C₃ alkyl)amino, —C(O)O-(C₁-C₃ alkyl), —C(O)NH-(C₁-C₃ alkyl), —CH═NOH, —PO₃H₂, —OPO₃H₂, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, alkylaryl, aralkenyl, aralkyl, alkylheterocyclyl, heterocyclylalkyl, sulfonyl, sulfonamido, carbamate, aryloxyalkyl, carboxyl and —C(O)NH(benzyl);
n is an integer of zero to four;
R² is selected from the group consisting of alkyl, alkenyl, alkynyl, alkoxy, alkenoxy, alkynoxy, aliphatic acyl, —C₁-C₃ aminoalkyl, aminoalkenyl, aminoalkynyl, di(C₁-C₃ alkyl) aminoalkyl, —C(O)O—(C₁-C₃ alkyl), —C(O)NH-(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, aminoaryl, biaryl, heterocyclyl, alkylaryl, aralkenyl, aralkyl, alkylheterocyclyl, heterocyclylalkyl, carbamate, aryloxyalkyl, carboxyl and —C(O)NH(benzyl); and,
R³ is selected from the group consisting of hydrogen, —OR⁶, —R¹³ and —NR⁷R⁸;
wherein R⁶ and R⁷ are chiral moieties;
R¹³ is a bicyclic chiral moiety;
R⁸ is selected from the group consisting of hydrogen, alkyl, alkenyl, alkynyl, alkoxy, alkenoxy, alkynoxy, aliphatic acyl, —C₁-C₃ alkylamino, alkenylamino, alkynylamino, di(C₁-C₃ alkyl) amino,—C(O)O-(C₁-C₃ alkyl), —C(O)NH-(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, heterocyclyl, alkylaryl, aralkenyl, aralkyl, alkylheterocyclyl, heterocyclylalkyl, carbamate, aryloxyalkyl, carboxyl and —C(O)NH(benzyl);
wherein R¹, R², R³, R⁴, R⁵, R⁶, R⁷, R⁸ and R¹³ are unsubstituted or substituted with at least one electron donating or electron withdrawing group,
and salts thereof;
with the proviso that when R² is —C(C₆H₅)₃, each R¹ or R⁴ and R⁵ are H, and R³ is —OR⁶, R⁶ is not —CH(CH₃)(C₂H₅);
and the proviso that when R² is —C(C₆H₅)₃, and Q is [chemical structure]
wherein n is 4 and R¹ is H, R³ is not H or R¹³ when R¹³ is (1R)-(+)-2,10-camphorsultam.

2. A compound of claim 1 further comprising derivatives of said compound selected from the group consisting of esters, carbamates, aminals, amides and optical isomers thereof.

3. The compound of claim 1 wherein said chiral moieties R⁶ or R⁷ are amines.

4. The compound of claim 1 of the formula [chemical structure]
wherein R² is selected from the group consisting of alkyl, alkenyl, alkynyl, alkoxy, alkenoxy, alkynoxy, aliphatic acyl, —C₁-C₃ aminoalkyl, aminoalkenyl, aminoalkynyl, di(C₁-C₃ alkyl) aminoalkyl, —C(O)O-(C₁-C₃ alkyl), —C(O)NH-(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, aminoaryl, biaryl, heterocyclyl, alkylaryl, aralkenyl, aralkyl, alkylheterocyclyl, heterocyclylalkyl, carbamate, aryloxyalkyl, carboxyl, and —C(O)NH(benzyl);
R³ is selected from the group consisting of hydrogen, —OR⁶, —R¹³ and —NR⁷R⁸;
wherein R⁶ and R⁷ are chiral moieties;
R¹³ is a bicyclic chiral moiety;
R⁸ is selected from the group consisting of hydrogen, alkyl, alkenyl, alkynyl, alkoxy, alkenoxy, alkynoxy, aliphatic acyl, —C₁-C₃ alkylamino, alkenylamino, alkynylamino, di(C₁-C₃ alkyl)amino,—C(O)O-(C₁-C₃ alkyl), —C(O)NH-(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, heterocyclyl, alkylaryl, aralkenyl, aralkyl, alkylheterocyclyl, heterocyclylalkylcarbamate, aryloxyalkyl, carboxyl and —C(O)NH(benzyl); and,
R⁹, R¹⁰, R¹¹ and R¹² are independently selected from the group consisting of hydrogen, halogen, hydroxyl, alkyl, alkenyl, alkynyl, alkoxy, alkenoxy, alkynoxy, thioalkoxy, aliphatic acyl, —CF₃, nitro, amino, cyano, —N(C₁-C₃ alkyl)-C(O)(C₁-C₃ alkyl), —C₁-C₃ alkylamino, alkenylamino, alkynylamino, di(C₁-C₃ alkyl)amino, —C(O)O-(C₁-C₃ alkyl), —C(O)NH-(C₁-C₃ alkyl), —CH═NOH, —PO₃H₂, —OPO₃H₂, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, alkylaryl, aralkenyl, aralkyl, alkylheterocyclyl, heterocyclylalkyl, sulfonyl, sulfonamido, carbamate, aryloxyalkyl, carboxyl and —C(O)NH(benzyl);
wherein R² R³, R⁶, R⁷, R⁸, R⁹, R¹⁰, R¹¹, R¹² and R¹³ are unsubstituted or substituted with at least one electron donating or electron withdrawing group,
and salts thereof,
with the proviso that when R² is —C(C₆H₅)₃, R⁹, R¹⁰, R¹¹ and R¹² are H, and R³ is —OR⁶, R⁶ is not —CH(CH₃)(C₂H₅);
and the proviso that when R² is —C(C₆H₅)₃, R⁹, R¹⁰, R¹¹ and R¹² are H, R³ is not H or R¹³ when R¹³ is (1R)-(+)-2,10-camphorsultam.

5. A compound of claim 4 further comprising derivatives of said compound selected from the group consisting of esters, carbamates, aminals, amides and optical isomers.

6. A compound of claim 4 wherein said chiral moieties R⁶ or R⁷ are amines.

7. A compound of claim 4 wherein R² is selected from the group consisting of alkyl and aryl; and R⁹, R¹⁰, R¹¹ and R¹² are each independently selected from the group consisting of hydrogen, alkyl, aryl and halogen.

8. N-((1S)-1-Phenylethyl)[(2S,1R)-2-(1-triphenylmethylimidazol-4-yl)cyclopropyl]carboxamide.
